(19) United States
(12) Patent Application Publication
(10) Pub. No.: US 2024/0209350 A1
(43) Pub. Date: Jun. 27, 2024

(54) HIGH COMPLEXITY MICROCOMPARTMENT-BASED INTERACTION SCREENING

(71) Applicant: European Molecular Biology Laboratory, Heidelberg (DE)

(72) Inventors: Christoph Merten, Lausanne (CH); Leonie Kolmar, Lausanne (CH); Alexis Louis Autour, Lausanne (CH); Yu Fen Samantha Seah, Heidelberg (DE); Anna Mei, Heidelberg (DE); Christian Heinis, Lausanne (CH)

(21) Appl. No.: 18/557,184

(22) PCT Filed: Apr. 26, 2022
(86) PCT No.: PCT/EP2022/061082

§ 371 Date: Oct. 25, 2023

(30) Foreign Application Priority Data
Apr. 26, 2021 (EP) 21170566.0

(51) Int. Cl. C12N 15/10

(52) U.S. Cl. CPC C12N 15/1072; C12N 15/1055; C12N 15/1065

(57) ABSTRACT

The invention regards a method for screening for interactions between two compounds provided in libraries. The invention in particular provides a method for screening interactions, such as a binding between two molecules or macromolecules, that is not limited to a classical screening by bait and prey setup, and therefore allows for a high-complexity screening of large candidate libraries, including screening for immunological interactions such as interactions between T-cells and antigen presenting cells, or B-cells and their antigenic targets.

### FIELD OF THE INVENTION

The invention regards a method for screening for interactions between two compounds provided in libraries. The invention in particular provides a method for screening interactions, such as a binding between two molecules or macromolecules, that is not limited to a classical screening by bait and prey setup, and therefore allows for a high-complexity screening of large candidate libraries, including screening for immunological interactions such as interactions between T-cells and antigen presenting cells, or B-cells and their antigenic targets.

### DESCRIPTION

Cell-signalling is governed by different interactions between binding partners. These interactions lead to the attainment of a function or communication between at least two entities. Here, the method describes a highly complexed approach for screening binding interactions of natural (e.g. proteins, antibodies) or synthetic (e.g. drugs) partners. In pharmaceutical drug development many different technologies have been established to screen highly complex libraries of biomolecules (such as antibodies, proteins, peptides or nucleic acids) or synthetic organic molecules in order to identify new therapeutics. One widely established method for screening high complexity libraries of drug candidates are so called DNA-encoding libraries (DEL), where a candidate compound of any nature is chemically labelled with a unique nucleic acid sequence, which are widely used in by the pharmaceutical industry for high throughput screening (HTS) (Song M et al J. Med. Chem. 2020, 63, 6578-6599).

Identifying binding interactions is an important task in biological and medical sciences. Interactions of proteins, nucleic acids with each other or chemical compounds or metabolites provide important clues about cellular signalling networks and metabolic and genetic regulation mechanisms. Moreover, inhibitors of such interactions are important pharmaceuticals, so there is a direct impact of the elucidation of biological interactions on medicine. Screening methods can be separated essentially into two groups: in the first group, interacting targets are identified as such, i.e. the identification method comprises a step wherein the information comprised in the protein or other molecule itself is used for identification. Examples for methods falling into this group are immunoprecipitation, surface plasmon resonance screening, or peptide-array screening methods. Identification of proteins is accomplished e.g. by sequence determination through Edman-degradation or mass-spectrometry of peptides derived from the protein.

In the second group, the protein and the sequence coding for it are coupled tightly, e.g. by maintaining both in close spatial proximity. In these assays, exemplified by phage display (Bratkovic T. (2010) “Progress in phage display: evolution of the technique and its application” Cell Mol Life Sci. March; 67(5):749-767) or the various types of two-hybrid screening methods (Dove and Hochschild (2004), “A bacterial two-hybrid system based on transcription activation”, Methods Mol Biol. 261:231-4; Brückner et al. (2009), “Yeast two-hybrid, a powerful tool for systems biology”, Int J Mol Sci. 10(6):2763-8; Lievens et al. (2009) “Mammalian two-hybrids come of age”, Trends Biochem Sci. 34(11):579-88; Lalonde et al. (2008), “Molecular and cellular approaches for the detection of protein-protein interactions: latest techniques and current limitations”, Plant J. 53(4):610-35), bacteriophages or cells containing interacting proteins are identified and the DNAs encoding said proteins are extracted. Identification of proteins is accomplished by DNA sequencing.

Younger et al. 2017 (Proceedings of the National Academy of Sciences of the United States of America, 114(46), 12166-12171) offer the possibility to screen two protein libraries and map an interaction network through a yeast display feature with chromosomal barcode and Next Generation Sequencing (NGS). Nonetheless, their methods furnish a protein-protein interaction only with a relatively small library and no other molecules.
Egloff et al. 2019 (Nature Methods, 16(5), 421-428) present three applications to screen protein-protein interaction using an in vitro approach without using phenotype-linkage. Instead, they are using genetically encoded barcoding peptides to retrieve the interacting proteins with liquid chromatography-tandem mass spectrometry (LC-MS/MS) and next generation sequencing analysis. The main limitation to this approach is the library size limiting to few thousand (˜10\hat{ }3 binders).

Known droplet-based microfluidic applications as shown for example in WO 2016/048994 are used with in vitro two-hybrid system (IVT2H) and one library to identify potential peptide binder. First, the droplets are generated using an initial microfluidic device to encapsulate one gene per droplet. After an incubation time, the droplets that contain a protein interaction will lead to the production of a fluorescent signal (e.g. GFP) and will be detected and sorted accordingly using a second microfluidic device. Besides the fact that only one small library can be screened, the microfluidic workflow is more complex and requires a consequent installation and know-how with the use of lasers and sorting.

Thermal proteome profiling (Mateus et al. 2020, Molecular Systems Biology, 16(3), 1-11) is a mass spectrometry based proteomic analysis method that can be used to identify protein interactions with small molecules, metabolites or other proteins. This method is based on the effect that proteins change their thermostability behaviour upon interacting with another molecule. The current biggest limitation of this approach is its low throughput due to the nature of mass spectrometry-based proteomics.

To date, screening assays of the second group are usually performed keeping one interaction partner constant (the “bait” protein), whereas the other interaction partner normally is provided as a library of candidate proteins (the “prey” proteins) that are expressed within a test-cell. Prey proteins interacting with the bait are identified by sequencing the nucleic acid sequences encoding the said prey proteins within a biological cell. Efficiency of such assays is limited since single clones have to be isolated to make sequencing possible. So, it is desirable to develop protein-protein interaction screening methods allowing for a more efficient screening and in particular a screening in higher complexity with respect of number of screened candidate interaction pairs.

### BRIEF DESCRIPTION OF THE INVENTION

Generally, and by way of brief description, the main aspects of the present invention can be described as follows:

In a first aspect, the invention pertains a method for the identification of at least two interacting entities comprised in two separated libraries of candidate interacting entities, the method comprising the steps of:
(a) Providing at least a first candidate entity library and a second candidate entity library each library comprising a plurality of candidate interacting entities each of which is composed of at least an interacting-portion and a labelling-portion, wherein one or more candidate interacting entities of the first candidate entity library are assumed to interact with one or more candidate interacting entities of the second candidate entity library (and vice versa);
(b) Bringing into contact the candidate interacting entities of the first candidate entity library with the candidate interacting entities of the second candidate entity library under conditions that allow for the formation of an interaction-complex between two interacting entities (or more interacting entities, preferably 2, or 3, or 4 or more);
(c) Encapsulating any entity and any interaction-complex from (b) in a plurality of microfluidic compartments under at least the conditions:
   A ratio of entities to compartments which is larger than 0 and less than 1 (preferably less than 0.1); and
   Optionally, a presence of one or more means for identifying of one or more labelling portion encapsulated within a compartment;
(d) Detecting subsequent to step (c) within the plurality of compartments, which comprise encapsulated entities, a presence of, and preferably an identity of, at least two labelling-portions encapsulated within a single compartment, wherein the presence of two labelling portions within a single compartment is indicative for an interaction between the two candidate interacting entities encapsulated within said compartment.

In a second aspect, the invention pertains to a method for screening for interaction between two entities each of which are comprised in a plurality of entities (library), the method comprising performing the steps of the method of the first aspect.

### DETAILED DESCRIPTION OF THE INVENTION

In the following, the elements of the invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

The invention solves the indicated problem in a first aspect by a method for the identification of at least two interacting entities comprised in two separated libraries of candidate interacting entities, the method comprising the steps of:
(a) Providing at least a first candidate entity library and a second candidate entity library each library comprising a plurality of candidate interacting entities each of which is composed of at least an interacting-portion and a labelling-portion, wherein one or more candidate interacting entities of the first candidate entity library are assumed to interact with one or more candidate interacting entities of the second candidate entity library (and vice versa);
(b) Bringing into contact the candidate interacting entities of the first candidate entity library with the candidate interacting entities of the second candidate entity library under conditions that allow for the formation of an interaction-complex between two interacting entities;
(c) Encapsulating any entity and any interaction-complex from (b) in a plurality of microfluidic compartments under at least the conditions:
   A ratio of entities to compartments which is larger than 0 and less than 1 (preferably less than 0.1); and
   Optionally, a presence of one or more means for identifying of one or more labelling portion encapsulated within a compartment;
(d) Detecting subsequent to step (c) within the plurality of compartments, which comprise encapsulated entities, a presence of, and preferably an identity of, at least two labelling-portions encapsulated within a single compartment, wherein the presence of two labelling portions within a single compartment is indicative for an interaction between the two candidate interacting entities encapsulated within said compartment.

The present invention seeks to identify interacting entities based on the idea that if an interaction complex of two interacting entities (molecules) is formed, such complex can be separated from non-interacting entities in close spatial proximity by encapsulation of the complex at very low entity concentration. The reduction of the concentration results in an encapsulation of on average less than one entity or interacting complex. In this scenario, non-interacting entities (e.g. phage particles displaying proteins that do not bind to any other within the library to be tested) will most likely end up in separate droplets, whereas entities interacting with each other (e.g. a phage particle displaying a protein drug target and another one displaying an antibody binding to it) will get encapsulated into the same droplet, despite their low concentration. This allows to specifically perform a fusion PCR (also known as overlap extension PCR) of the genes encoding interacting entities when using e.g. a forward primer binding to the backbone encoding all members of library one (e.g. the antibody library) and a reverse primer binding to the backbone encoding all members of library two (e.g. open reading frames of all proteins of a pathogen as potential drug targets). By using specifically adapted screening libraries which comprise entities coupled to certain labelling moieties, such as nucleic acids, an identification of the interacting entities can be performed in an additional step. While the present invention is exemplified using nucleic acid-based hybridization and amplification techniques for the final step of determining entity identity, the invention shall not necessarily be restricted to nucleic acid-based methods. Primer selection for such fusion PCR allows for the generation of a PCR product that combines the sequences of the labelling portions of both libraries. For each library, the labelling portion of each entity comprises a sequence that is specific to the individual entity, and a further (outer) sequence specific for the library the entity is contained in. This allows a use of two separate primer pairs of which each amplify only a labelling portions of an entity of one library. During fusion PCR, at least one of each primer of each set comprises an overlapping section complementary with an overlapping section of one primer used for the amplification in the other library. Therefore, during PCR a larger PCR product can be generated (see FIG. 1). By using during the fusion PCR procedure, a set of primers that comprise a hybridization element specific for each library identifying sequence. Such overlapping primer section can be introduced into either the “inner” or “outer” primers.

In a preferred embodiment of the invention can also be applied to screen for binding-interaction between cells. In this particular embodiment the candidate entity of one or more candidate entity libraries of the invention is a cell. In this embodiment the invention seeks to screen for interactions between cells and another entity or between cells. Such cell-based interactions including cell-to-cell interactions, are based on cell-surface based interaction components, such as receptor-ligand interactions, which mediate very specific cell mediated interactions. Of particular interest are such cell-based screens, including cell-to-cell interaction screens, in immunology, for example for the development of T-cell based therapeutics. For example, the invention comprises an interaction screen for the identification of matching pairs of T-cell receptors (TCRs, displayed on T-cells) and antigens (peptide antigens displayed on the MHC2 complex of antigen presenting cells, APCs). In particular, the invention therefore pertains to a method as described herein before, wherein the candidate entity libraries are cell libraries comprising a plurality of cells presenting a variety of different MHC bound peptides on the one hand, and a second candidate entity library comprising cells each expressing a particular T-cell receptor clone. In such an embodiment it is preferred that the antigenic peptide is loaded on to the APC.

The term “antigen presenting cell” includes a B cell, dendritic cell, macrophage, activated epithelial cell, fibroblast, thymic epithelial cell, thyroid epithelial cell, glial cell, pancreatic beta cell, and a vascular endothelial cell. In some embodiments, the APC is a professional APC such as a dendritic cell, macrophage, B cell, or an activated epithelial cell. In other embodiments, the APC is a non-professional APC such as a fibroblast, thymic epithelial cell, thyroid epithelial cell, glial cell, pancreatic beta cell, or a vascular endothelial cell. Since cancer cells also act as antigen presenting cells, the term “antigen presenting cell” further includes cancer cells.

The term “MHC”, may include both MHC or HLA class I and MHC or HLA class II complexes. Similarly the invention pertains both to CD4 and CD8 positive T cells, depending on the specific interaction to be screened.

The present invention surprisingly provides a further screening method for the identification of a specific immunological cell-to-cell interactions, such as an interaction between a T-cell receptor and an MHC-presented antigenic peptide. The surprising aspect of the invention lies in the fact that the interaction between TCR expressing T-cells and antigen presenting cells (APCs) is detectable as duplet formation (or two-entity complexes), and therefore can be subjected to the screening method of the invention comprising compartmentalization of the cell duplets. In a similar embodiment the invention can be used to detect an interaction between B-cell receptor expressing B-cells and a target antigen (and/or cell surface expressed antigen).

Thus, in some preferred embodiments of the invention the first candidate entity library is a library of T-cell, wherein each T-cell comprises one distinct and rearranged T-cell receptor gene encoding for a specific T-cell receptor. The second candidate entity library is a library of antigen presenting cells, wherein each antigen presenting cell comprises on its surface an MHC complex presenting an antigenic peptide with a specific sequence.

In one alternative embodiment, one of the candidate entity libraries is a B-cell library expressing one distinct and rearranged B-cell receptor gene encoding for a specific B-cell receptor. In this embodiment, the second library can be selected from a library of candidate soluble antigens, or cells expressing cell surface located candidate antigens.

In some embodiments, the labelling portion which is a nucleic acid may comprise a hybridization portion that is specific for a certain library of entities. Such hybridization portions in this embodiment preferably comprise a sequence which is complementary to, or hybridizes under stringent conditions to, a second hybridization portion comprised in a labelling portion of entities of a second, and different library. In such screening approaches an interaction of the entities of both libraries to one another may be screened. If interacting entities are encapsulated, the hybridization portions of both candidate interacting entities can hybridize and form a fused template for a fusion PCR.

In one further embodiment, the labelling portions which are nucleic acids may be simply ligated together, either blunt or using short overhangs. In some embodiments, the primer which is not used for linking (overlap) may in addition comprise an outer hybridization element that can be used for a second PCR in order to exponentially amplify the fused PCR product.

More generally the term “fusion PCR” or “overlap extension PCR” means that the PCR products are formed into overlapping chains by using primers having complementary ends, thereby overlapping the amplified fragments of different sources by overlapping extension chains in subsequent amplification reactions. In certain preferred embodiments of the invention, if a nested PCR is performed to amplify a fused PCT product, the amplification reaction is performed with a higher concentration of outer primers compared to the inner primer pair (such as preferably 2 fold, 3 fold, 4 fold, 6 fold, 8 fold or 10 fold, 100 fold or higher). This embodiment allows for a stronger amplification of the fused product compared to possible shorter non-interaction dependent amplification products.

A ratio of entities to compartments in step (c) is in certain embodiments larger than 0 and less than 1, preferably which is larger than 0 and in increasing preference less than 0.5, 0.2, 0.1, 0.05, most preferably less than 0.01.

An “interacting entity” in context of the invention shall be any molecule or molecule complex, which can form a non-covalent or covalent interaction with another interacting entity. In preferred embodiments of the present invention the interacting entities form specific and selective interactions with each other. A typical example of interacting entity pairs in accordance with the present invention are interaction pairs such as antibody-antigen, nucleic acid hybridization, receptor-ligand, enzyme-substrate, small molecule inhibitor and target protein, etc.

An entity library in accordance with the present invention comprises a plurality of distinct single entities. The entity library can have from 10 to 10⁹ candidate entity members, e.g., from 10 candidate entities to 10² candidate entities, from 10² candidate entities to 10³ candidate entities, from 10³ candidate entities to 10⁴ candidate entities, from 10⁴ candidate entities to 10⁵ candidate entities, from 10⁵ candidate entities to 10⁶ candidate entities, from 10⁶ candidate entities to 10⁷ candidate entities, from 10⁷ candidate entities to 10⁸ candidate entities, or from 10⁸ candidate entities to 10⁹ candidate entities. In some cases, the library has more than 10⁹ candidate entities.

In a preferred embodiment of the invention protein-protein interaction partners are screened with the inventive method.

In certain particular embodiments of the invention, which may be preferred, the method of the invention comprises a purification step between steps (b) and (c) in order to remove non-interacting entities which are not in an interaction complex. In this embodiment, the candidate interacting entities of the libraries further comprise purification tags which are representative for their library. Including a step of purification using the purification tag of a given entity library, entities of the other libraries which are not within an interaction-complex will be discriminated against in the purification, and their fraction is accordingly reduced. In addition, the method may therefore comprise a purification step for each species of entity library used in the method. Such multiple purification steps are done in sequence and preferably not concomitantly. Furthermore, in this embodiment the ratio of entities to compartments in step (c) is preferably 0, 1 or larger, and less than 1; more preferably is larger than 0.1 and less than 1, for example is about 0.5.

For example, a preferred entity library of the invention is a library of molecules attached to a nucleic acid labelling portions, also known as DNA-encoded library technology (DELT). A DNA-encoded library (DEL) is composed of a pool of different molecules, each being a conjugate between a small organic molecule and a specific DNA sequence (a so-called “DNA barcode” which shall be understood to constitute a labelling portions in context of the present invention), thus realizing a direct physical connection between function, such as function of the candidate entity molecule by its chemical structure as an interacting entity) and information (information about the type of small organic molecule coded by the DNA sequence). The DNA sequences are designed to identify the associated chemical structures of the candidate entity using various technologies, e.g. Sanger sequencing, DNA array and/or high throughput—next generation—sequencing. Candidate entities used in DELs may range from small or large organic molecules to biomolecules such as proteins, sugars, nucleic acids, fatty acids, or any combination of the forgoing.

Preferably, in some embodiments the method of the present invention is performed extra-cellular, in other words, the screening steps (a) to (d) are performed without performing a protein expression and/or presentation within a biological cell.

Further, an interaction between two interacting entities according to the invention is in preferred embodiments a “binding” between the two interacting entities. The term “binding” refers to a direct association between two entities such as molecules (e.g., two polypeptides of a protein-protein interaction pair), due to, for example, covalent, electrostatic, hydrophobic, and ionic and/or hydrogen-bond interactions, including interactions such as salt bridges and water bridges. A “specific binding” refers to binding with an affinity between two protein interaction entities of at least about 10⁻⁷ M or greater, e.g., 5×10⁻⁷ M, 10⁻⁸ M, 5×10⁻⁸ M, and greater. Contrary thereto, a “non-specific binding” refers to binding with an affinity of less than about 10⁻⁷ M, e.g., binding with an affinity of 10⁻⁶ M, 10⁻⁵ M, 10⁻⁴ M, etc. In some cases, e.g., in instances of transient protein-protein interactions, “specific binding” can be lower than 10⁻⁷ M; e.g., specific binding can be binding with an affinity of at least 10⁻⁵ M or greater, e.g., 10⁻⁵ M, 10⁻⁶ M, or 10⁻⁷ M. Binding affinities can depend on the chemical environment, e.g. the pH value, the ionic strength, the presence of co-factors, etc. In the context of the present disclosure, the term “protein-protein interaction” can refer to protein-protein interactions occurring under physiological conditions, i.e. under conditions found in a living cell.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

In some cases, the first and the second members of the first candidate entity library and a second candidate entity library are naturally-occurring polypeptides. In some cases, one or both of the first and the second members of the candidate libraries is a non-naturally-occurring polypeptide, e.g., a recombinant polypeptide made in the laboratory, or mutated compared to a naturally-occurring polypeptide. In some cases, the first member of the protein interaction pair is an N-terminal portion of a polypeptide; and the second member of the protein interaction pair is a C-terminal portion of the polypeptide. In some cases, the first member of the protein interaction pair is a known protein; and the second member of the protein interaction pair is an unknown protein, e.g., a member of a library of proteins. In some cases, the first member of the protein interaction pair is a first known protein that binds to a second known protein, and the second member of the protein interaction pair is a variant of the second known protein.

As such, in certain alternative aspects, the first or second candidate entity library may comprise a limited number of members, even only one known candidate interacting entity. For example, in some cases, the first member of an interaction pair to be screened (which first member may be referred to as a “bait”) is a small selection of known polypeptides; and the second member of the interaction pair (which second member may be referred to as a “prey”) is a member of a library of proteins (e.g., a plurality of proteins) of unknown amino acid sequence and/or function. The known interaction partner can be any of a variety of selection of molecules, for example for proteins they may include membrane proteins, receptors, enzymes, cytoskeletal proteins, regulatory proteins, transcription factors, and the like. The unknown protein can be a member of a candidate compound library, where the compound library can have from 10 to 10⁹ members, e.g., from 10 members to 10² members, from 10² members to 10³ members, from 10³ members to 10⁴ members, from 10⁴ members to 10⁵ members, from 10⁵ members to 10⁶ members, from 10⁶ members to 10⁷ members, from 10⁷ members to 10⁸ members, or from 10⁸ members to 10⁹ members. In some cases, the library has more than 10⁹ members.

The interacting-portion of the invention may be selected from any molecular entity of interest including and includes a candidate entity which may be one selected from a polypeptide, peptide, glycoprotein, a peptidomimetic, an antigen binding construct (for example, an antibody, antibody-like molecule or other antigen binding derivative, or an or antigen binding fragment thereof), a nucleic acid such as a DNA or RNA, for example an antisense or inhibitory DNA or RNA, a ribozyme, an RNA or DNA aptamer, RNAi, siRNA, shRNA and the like, including variants or derivatives thereof such as a peptide nucleic acid (PNA).

In particular embodiments of such screening method, the candidate entity is a small (organic) molecule of any kind. Typically, a small molecule is a compound having a molecular mass of less than about 750 Da, such as less than about 650 or 600 Da, (and in certain embodiments, a small molecule may be less than about 550 or 500 Da).

In preferred embodiments, the interacting-portion is a part of a macro-molecule or molecule complex that mediates the interaction for which a screening according to the invention is performed. Usually, the interacting-portion is a proteinaceous molecule, or is a polypeptide or a section or domain of a polypeptide, such as a domain known to mediate an interaction to other entities. Such an interacting domain may be known to mediate protein-protein interactions such as immunoglobulin domains, or can be a domain comprising an active site of enzyme and known to bind small molecular substrates etc. In addition, also small organic or inorganic compound libraries comprising labelled small molecules, for example labelled with nucleic acids, may be used for a screening in accordance with the present invention. Mixed approaches where one library is for example a protein library and the other a library is composed of small molecules, for example potential inhibitors of a protein, are in particular encompassed by the present invention.

In context of the invention in order to allow the identification of the presence of two interacting entities within a compartment subsequent to encapsulation, a “labelling portion” is necessary. The labelling portion may be any molecular entity that allows for a determination of the presence or absence of the candidate entity. In preferred embodiments, the labelling portion allows in addition for the identification of the entity—in such case, the labelling portion may also be referred to as a “barcoding portion”, or a “barcode”. Instances where nucleic acid-based barcode identification and/or quantification is performed by sequencing, including e.g., Next Generation Sequencing methods, conventional considerations for barcodes detected by sequencing will be applied. In some instances, commercially available barcodes and/or kits containing barcodes and/or barcode adapters may be used or modified for use in the methods described herein, including e.g., those barcodes and/or barcode adapter kits commercially available from suppliers such as but not limited to, e.g., New England Biolabs (Ipswich, Mass.), Illumina, Inc. (Hayward, Calif.), Life Technologies, Inc. (Grand Island, N.Y.), Bioo Scientific Corporation (Austin, Tex.), and the like, or may be custom manufactured, e.g., as available from e.g., Integrated DNA Technologies, Inc. (Coralville, Iowa). Barcode length will vary and will depend upon the complexity of the candidate entity library and the barcode detection method utilized. As nucleic acid barcodes (e.g., DNA barcodes) are well-known, design, synthesis and use of nucleic acid barcodes is within the skill of the ordinary relevant artisan.

Therefore, in embodiments where a nucleic acid barcode is used, preferably each distinct candidate interacting entity comprises a nucleic acid molecule having at least one identification-sequence which is unique to the interacting-portion of the distinct candidate interacting entity. Preferably, it is known which sequence of the barcode codes for which interacting portion (or candidate interacting entity), and therefore, detection of the presence of a unique barcode sequence indicates the presence of and identity of a unique interacting portion. A “barcode sequence” in the present context preferably relates to a nucleic acid sequence allowing for an unambiguous identification of the interaction portion having said barcode sequence. Preferably, a barcode sequence consists of a sequence of at least ten, at least eleven, at least twelve, at least thirteen, at least fourteen, at least fifteen, at least sixteen, at least eighteen, at least twenty consecutive randomly assembled nucleotides. Preferably, said barcode sequence is theoretically unique. It is well known in the art how random sequences can be achieved in oligonucleotide synthesis. It is to be understood that the number of different polynucleotide molecules theoretically possible is directly dependent on the length of the barcode sequence; e.g., if a DNA barcode with randomly assembled Adenine, Thymidine, Guanosine and Cytidine nucleotides is used, the theoretical maximal number of barcode sequences possible is 1,048,576 for a length of ten nucleotides, and is 1,073,741,824 for a length of fifteen nucleotides. Preferably, the length of the barcode sequences is selected such that the number of unique sequences theoretically possible is at least as high as the number of preys used in a pool of sequences. The person skilled in the art knows how to adopt the length of the barcode sequence. Preferably, said barcode sequences are inserted into a pre-defined nucleotide sequence, e.g. a restriction enzyme recognition site, such that the start point of said barcode sequence is pre-determined unambiguously. Most preferably the identification sequence or barcode sequence comprises a nucleic acid sequence encoding at least parts of the amino acid sequence of the proteinaceous interacting-portion.

In particular embodiments of the invention, the identification sequence is flanked by an upstream primer binding sequence and a downstream primer binding sequence, which both are different and do not anneal to each other during an annealing phase of a PCR amplification cycle. Using such primer binding sequences, the method may include an amplification step in order to detect the presence and identity of a barcode sequence. For example, in some embodiments, the method of the invention may comprise that step (d) involves a PCR amplification, preferably a fusion PCR and wherein the means for identifying of one or more labelling portion comprises components sufficient for conducting the PCR amplification, preferably the fusion PCR. As used herein, the term “fusion PCR” refers to PCR methodology which is used to join or fuse a plurality of polynucleotide fragments into a conjoined polynucleotide fragment.

Such “means for identifying of one or more labelling portion” in preferred embodiments of the invention comprise a first and a second PCR primer pair, wherein the upstream primer of the first PCR primer pair anneals to the upstream primer binding sequence of each labelling-portion contained in the first candidate entity library, and the downstream primer of the first PCR primer pair anneals to the downstream primer binding sequence of each labelling-portion contained in the first candidate entity library; and wherein the upstream primer of the second PCR primer pair anneals to the upstream primer binding sequence of each labelling-portion contained in the second candidate entity library, and the downstream primer of the second PCR primer pair anneals to the downstream primer binding sequence of each labelling-portion contained in the second candidate entity library.

A “primer binding sequence” as used herein relates to a nucleic acid sequence known to specifically hybridize to a predefined PCR primer under conditions typically used in PCR or other polynucleic acid amplifying methods. Thus, the primer binding sequence of the current invention consists of at least fifteen, at least sixteen, at least seventeen, at least eighteen consecutive nucleotides with a known sequence. Preferably, the polynucleotide of the invention comprises two primer binding sequences, wherein the melting temperature of the primer binding sequences differs by no more than six, no more than five, no more than four, no more than tree, or no more than two degrees Celsius. More preferably, the first and the second primer binding sequence differ in their nucleotide sequence to such an extent that an oligonucleotide specifically hybridizing to the first primer binding sequence does not hybridize specifically to the second primer binding sequence and that a primer specifically hybridizing to the second primer binding sequence does not hybridize specifically to the first primer binding sequence.

As used herein, the term “flanked” means being arranged in close proximity. Preferably, the barcode sequence of the current invention is flanked by a first and a second primer binding sequence such that a nucleic acid produced by PCR using primers hybridizing specifically to said first and second primer binding sequences will consist of no more than 300, no more than 250, no more than 200, no more than 150, no more than 100, no more than 75, or no more than 50 nucleotides. More preferably, the first and the second primer binding sequence are separated from the barcode sequence by no more than ten, eight, six, five, four, three, or two nucleotides.

In some embodiments of the invention, each primer binding sequence of the labelling portion of the candidate interacting entity of the first candidate entity library differs from each primer binding sequence of the labelling portion of the candidate interacting entity of the second candidate entity library (and vice versa).

In embodiments, wherein a fusion PCR is performed, it is preferable that the upstream- and the downstream primer of the first primer pair comprises a first cross-hybridization sequence and the upstream- and the downstream primer of the second primer pair comprises a second cross-hybridization sequence; wherein the first- and the second cross-hybridization sequence hybridize to each other under annealing conditions during a PCR annealing step. Hence, a PCR amplification in step (c) may comprise a fusion PCR immediately followed by the removal of residual primer oligonucleotides and the subsequent nested PCR using (i) an upstream primer which anneals to the upstream primer binding sequence of each labelling-portion contained in the first candidate entity library, and a downstream primer which anneals to the downstream primer binding sequence of each labelling-portion contained in the second candidate entity library; or (ii) an upstream primer which anneals to the upstream primer binding sequence of each labelling-portion contained in the second candidate entity library, and a downstream primer which anneals to the downstream primer binding sequence of each labelling-portion contained in the first candidate entity library; wherein step (d) involves the detection of the amplification product of the nested PCR and wherein the presence of an amplification product indicates the presence of two labelling portions within a single compartment.

The amplification product so produced allows therefore the detection of the interaction, and, if sequenced, in some embodiments also the identification of the interacting entities. Hence, the method of the invention may further comprise a step of sequencing the amplification product of the nested PCR in order to determine the identity of the interacting-portions which were comprised within one compartment.

The invention, in some embodiments, may also be realized using an indirect labelling of individual entities in one or more candidate entity libraries. For example, the invention pertain to candidate entities wherein the labelling portion is a binding portion, such as an amino acid epitope, which allows for a specific binding of a labelling entity to the candidate entity. In this embodiment, the labelling entity comprises a binding portion mediating the binding to the candidate entity, and a labelling portion, such as a nucleic acid-based label or barcode, which then allows for a detection of an interaction of two or more candidate entities. A binding interaction between a candidate entity and a labelling entity may be based on any specific and/or selective protein-protein or protein-nucleic acid interactions known in the art, but preferably include protein epitope tag based technologies selected from the list of interaction partners including but not limited to: antibodies, and any derivatives, variants or fragments thereof, such as nanobodies, or single-chain fragments (scFv, or scFab), nucleic acid aptamers, and similar proteins, ligand-receptor based binding, including T-cell receptor antigenic peptide interactions, or major histocompatibility complex (MHC)-antigenic peptide based interactions.

In preferred embodiments of the invention the candidate interacting entities are provided as protein conjugates comprising a polypeptide sequence (or protein fragment) as interacting portion covalently fused to a nucleic acid sequence which constitutes the labelling portion. As an alternative embodiment, the invention may also be realized using a phage display library as candidate interacting entity library. In the latter case the interacting portion is provided as a protein presented on the phage coat, and the labelling portion can be a nucleic acid encapsulated within the phage.

Further examples useful for the present invention for the presentation of the candidate interacting entities is the use of mRNA display, yeast display, retroviral display. The term “mRNA display” is an in vitro selection technique used to obtain from libraries of diverse sequences peptides and proteins that have an affinity for a target ligand/material. The process relies on mRNA-protein fusion molecules, which consist of peptide or protein sequences covalently linked via their C-termini to the 3′ end of their own mRNA. Yeast display is based on presenting protein variants on the surface of yeast cells. Each cell typically displays only one protein variant of a library, whose identity is encoded in a specific gene sequence expressed in the corresponding cell (coupling of genotype and phenotype). Retroviral display follows the same principles, but the proteins are displayed on the retrovirus membrane and the gene encoding a particular variant is encapsulated in form of an RNA transfer gene in the corresponding particle.

Preferably, the method is performed by using encapsulation into compartments such as any droplet, particle or well, and preferably micro compartments such as into droplets that can be used in a microfluidic system.

The term “microfluidic droplet” refers to an aqueous microcompartment of a certain size that encapsulates an aqueous liquid. The size of the microfluidic droplet is usually expressed as the diameter of the droplet when in a spherical shape. The diameter is generally between 1 and 500 μm, or between 20 and 400 μιη, and preferably between 30 and 350 μιη, between 40 and 300 μιη, between 40 and 250 μιη, between 40 and 200 μιη or between 40 and 100 μιη (wherein each narrower range is preferred to the foregoing broader ranges and “between” includes the values mentioned). In a preferred embodiment, the diameter of the microfluidic droplet is between 2 and 20 times the diameter of the largest particle (e.g. the first particle or a further particle) in the droplet, preferably between 3 and 18 times, between 4 and 16 times, or between 5 and 14 times or between 6 and 12 times (wherein each narrower range is preferred to the foregoing broader ranges and “between” includes the values mentioned). Preferably, the diameter of the droplet is defined by both the above absolute and relative parameters. For example, the diameter is (i) between 20 and 400 μιη, between 30 and 350 μιη, between 40 and 300 μιη, between 40 and 300 μιη, between 40 and 250 μιη, between 40 and 200 μιη or between 40 and 100 μιη and between 2 and 20 times the diameter of the largest particle (e.g. the first particle or a further particle) in the droplet, (ii) between 20 and 400 μιη, between 30 and 350 μιη, between 40 and 300 μιη, between 40 and 300 μιη, between 40 and 250 μιη, between 40 and 200 μιη or between 40 and 100 μιη and between 4 and 16 times the diameter of the largest particle (e.g. the first particle or a further particle) in the droplet, or (iii) between 20 and 400 μιη, between 30 and 350 μιη, between 40 and 300 μιη, between 40 and 300 μιη, between 40 and 250 μιη, between 40 and 200 μιη or between 40 and 100 μιη and between 6 and 12 times the diameter of the largest particle (e.g. the first particle or a further particle) in the droplet.

Alternatively, the size of the microfluidic droplet can also be defined by volume. For example, it is usually less than 1 microlitre (μl). Preferably, it is less than 500 nanolitres (nl), less than 250, less than 150, less than 100 or less than 50 nl or less, such as less than 1 nl, or less than 100 pl or less. In a preferred embodiment, it is between 0.05 and 150 nl, preferably between 0.05 and 125 nl, between 0.05 and 100 nl, between 0.05 and 80 nl, or between 0.05 and 4 nl (wherein each narrower range is preferred to the foregoing broader ranges and “between” includes the values mentioned). For screening setups with droplets in the smaller μm range, volumes of less than 1 pl. Microdroplets of the invention for screening purposes have a volume of less than 1 pl, preferably of 01, or even 0.01 pl.

A wide variety of compartmentalisation or microencapsulation procedures are available (Benita, S., Ed. (1996). Microencapsulation: methods and industrial applications. Drugs and pharmaceutical sciences. Edited by Swarbrick, J. New York: Marcel Dekker) and may be used to create the microfluidic droplet used in accordance with the present invention. Indeed, more than 200 microencapsulation or compartmentalisation methods have been identified in the literature (Finch, C. A. (1993) Encapsulation and controlled release. Spec. Publ.-R. Soc. Chem. 138, 35). These include membrane enveloped aqueous vesicles such as lipid vesicles (liposomes) (New, R. R. C, Ed. (1990). Liposomes: a practical approach. The practical approach series. Edited by Rickwood, D. & Hames, B. D. Oxford: Oxford University Press) and non-ionic surfactant vesicles (van Hal, D. A., Bouwstra, J. A. & Junginger, H. E. (1996). Nonionic surfactant vesicles containing estradiol for topical application. In Microencapsulation: methods and industrial applications (Benita, S., ed.), pp. 329-347. Marcel Dekker, New York). Preferably, the microcompartments of the present invention are formed from emulsions; heterogeneous systems of two immiscible liquid phases with one of the phases dispersed in the other as droplets of microscopic size (Becher, P. (1957) Emulsions: theory and practice. Reinhold, New York; Sherman, P. (1968) Emulsion science. Academic Press, London; Lissant, K. J., ed Emulsions and emulsion technology. Surfactant Science New York: Marcel Dekker, 1974; Lissant, K. J., ed. Emulsions and emulsion technology. Surfactant Science New York: Marcel Dekker, 1984). Emulsions may be produced from any suitable combination of immiscible liquids. Preferably the emulsion of the present invention has water (containing a particle and other components) as the phase present in the form of droplets and a hydrophobic, immiscible liquid (preferably an oil) as the surrounding matrix in which these droplets are suspended. Such emulsions are termed ‘water-in-oil’. This has the advantage that the aqueous phase is compartmentalised in discrete droplets. The external phase, preferably being a hydrophobic oil, generally is inert. The emulsion may be stabilized by addition of one or more surface-active agents (surfactants). These surfactants act at the water/oil interface to prevent (or at least delay) separation of the phases. Many oils and many emulsifiers can be used for the generation of water-in-oil emulsions; a recent compilation listed over 16,000 surfactants, many of which are used as emulsifying agents (Ash, M. and Ash, I. (1993) Handbook of industrial surfactants. Gower, Aldershot). Suitable oils are listed below.

In some specific embodiments of the invention, an interaction between two candidate interacting entities may be inducible under certain conditions, including the presence of an interaction inducing small molecular agent (a binding inducing agent).

In a particular further embodiment or aspect, the invention pertains a method for the identification of at least two interacting entities comprised in two separated libraries of candidate interacting entities, the method comprising the steps of:
(a) providing at least a first candidate entity library and a second candidate entity library each library comprising a plurality of candidate interacting entities each of which is composed of at least an interacting-portion and a labelling-portion, wherein one or more candidate interacting entities of the first candidate entity library are assumed to interact with one or more candidate interacting entities of the second candidate entity library (and vice versa); and wherein each interacting entity of the first entity library comprises a first purification tag not present in the second entity library, and/or wherein each interacting entity of the second entity library comprises a second purification tag not present in the first entity library;
(b) bringing into contact the candidate interacting entities of the first candidate entity library with the candidate interacting entities of the second candidate entity library under conditions that allow for the formation of an interaction-complex between two interacting entities (or more interacting entities, preferably 2, or 3, or 4 or more);
(b′) at least one purification step comprising purifying any entity and any interaction-complex from (b) using the first purification tag and/or the second purification tag to obtain purified mixture which is characterized by comprising an increased fraction of interaction-complexes;
(c) encapsulating any purified entity and any interaction-complex from the purified mixture in (b) in a plurality of microfluidic compartments under at least the conditions:
   A ratio of entities to compartments which is larger than 0 and less than 1 (and preferably is equal to 0.1, or larger than 0.1 and less than 1); and
   Optionally, a presence of one or more means for identifying of one or more labelling portion encapsulated within a compartment;
(d) detecting subsequent to step (c) within the plurality of compartments, which comprise encapsulated entities, a presence of, and preferably an identity of, at least two labelling-portions encapsulated within a single compartment, wherein the presence of two labelling portions within a single compartment is indicative for an interaction between the two candidate interacting entities encapsulated within said compartment.

The term “purification tag” as used herein refers to any molecule, or macromolecule (such as peptides, proteins, antibodies and derivatives or fragments thereof, or nucleic acid based tags) suitable for purification or identification of a candidate entity comprising the purification tag. The purification tag specifically binds to another moiety with affinity for the purification tag. Such moieties which specifically bind to a purification tag are usually attached to a matrix or a resin, such as agarose beads, used for, for example, column-based purification. Moieties which specifically bind to purification tags include antibodies, other proteins (e.g. Protein A or Streptavidin), nickel or cobalt ions or resins, biotin, amylose, maltose, and cyclodextrin. Exemplary purification tags include histidine (HIS) tags (such as a hexahistidine peptide), which will bind to metal ions such as nickel or cobalt ions. Other exemplary purification tags are the myc tag, the Strep tag, the Flag tag and the V5 tag. Preferably, the purification tag is selected from the group consisting of a polyhistidine tag, a polyarginine tag, glutathione-S-transferase (GST), maltose binding protein (MBP), influenza virus (HA) tag, thioredoxin, staphylococcal protein A tag, the FLAG™ epitope, and the c-myc epitope. The term “purification tag” also includes “epitope tags”, i.e. peptide sequences which are specifically recognized by antibodies. Exemplary epitope tags include the FLAG tag, which is specifically recognized by a monoclonal anti-FLAG antibody. In some embodiments, the polypeptide domain fused to the polymerase comprises two or more tags, such as a SUMO tag and a STREP tag. The term “purification tag” also includes substantially identical variants of purification tags. “Substantially identical variant” as used herein refers to derivatives or fragments of purification tags which are modified compared to the original purification tag (e.g. via amino acid substitutions, deletions or insertions), but which retain the property of the purification tag of specifically binding to a moiety which specifically recognizes the purification tag. Alternative purification tags may include nucleic acid-based tags, such as single stranded nucleic acid sequences that can be bound by their complementary antisense strand.

In a particular embodiment of this alternative, wherein each interacting entity of the first entity library comprises a first purification tag not present in the second entity library, and wherein each interacting entity of the second entity library comprises a second purification tag not present in the first entity library; and wherein the purification step (b′) includes two separate purification steps, wherein a first purification is performed using the first purification tag, and wherein subsequently (and not concomitantly) a second purification is performed using the second purification step. In this embodiment, the first step is performed in order to reduce the fraction if non-interacting entities comprising the second purification step, and wherein the second purification is performed in order to reduce the fraction of non-interacting entities comprises the first purification tag.

A “purification” in context of the invention shall therefore comprise a step wherein the entities, or complexes thereof, during purification are brought into contact with a capturing means which specifically bind the purification tag. Such capturing means, as mentioned above, may be for example a matrix material coupled to the capturing means and thereby allowing affinity-based purification.

In this particular embodiment, and as described elsewhere herein, a step of purification allows a use of a less stringent ratio of entities to compartments which is larger than 0 and less than 1 (preferably less than 0.1). A less stringent ratio in preferred embodiments is a ratio of between 0.1 and 1, preferably between 0.2 and, more preferably of between 0.3 and 1, lower than

In a second aspect, the invention pertains to a method for screening for interaction between two entities each of which are comprised in a plurality of entities (library), the method comprising performing the steps of the method of the first aspect.

In preferred embodiments of the invention, the methods of the first and second aspect of the invention are used for screening an interaction that has a therapeutically or diagnostic relevance. For example, the methods of the invention may be used to identify an interaction entity that interacts with a known entity or group of entities, and wherein the interaction entity can be used as a therapeutic.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES

The figures show:

FIG. 1: shows the general workflow of protein library against library screening. Two libraries of interaction partners that are each physically linked to its self-encoding nucleic acid are incubated together. Subsequently, the interaction partners are encapsulated in water in oil droplets with an occupancy corresponding to less than one interaction partner per droplet volume. Thus, non-interacting entities will most likely end up in separate droplets whereas interacting partners will be encapsulated in the same droplet. During a PCR that is performed in the droplets the DNA strands encoding for the interacting partners are fused together and specific primer sides are introduced at both ends. During a second PCR that is performed after breaking the emulsion only fused fragments of interacting partners can be amplified whereas DNA fragments of entities that were encapsulated alone are not amplified, or only amplified non-exponentially via a single PCR primer as they lack the necessary primer binding sites. In the last step interacting partners are identified by high-throughput sequencing of fused fragments

FIG. 2: shows an implementation of the workflow shown in FIG. 1 using phage display (e.g. an antibody library and a protein target library).

FIG. 3: shows a model system of genetically-encoded interaction partners made of oligonucleotides functionalized with click chemistry moieties (DBCO and Azide). Upon contact and incubation, these functional groups from a covalent bond.

FIG. 4: shows the overview of the workflow and PCR amplification steps of a Proof of Concept test of the invention. For the in vitro interaction model system, a copper-free click chemistry approach based on a strain-promoted alkyne azide cycloaddition (SPAAC) was applied. The sequences were chemically modified, either with a DBCO- or an azide group. Upon incubation and interaction, the modifications on the sequences formed a covalent bond (step 1). With the “clicked” fragments, a fusion PCR in droplets was performed, where oligonucleotides integrating linker and common sequences were used (step 2). Next, the emulsion was broken and the fused fragment were size selected (step 3, 4). Subsequently, a nested PCR was carried out to enrich for the fused fragment (step 5). Lastly, for determining which sequences were fused together, a qPCR with specific primer pairs was done (step 6). Same arrow types represent primers amplifying specifically each individual sequence or fused fragment, specific for every combination of sequences.

FIG. 5: shows the result of the Proof-of-Concept results for the in vitro interaction model system, testing two pairs (Pair A and B) each consisting of two different sequences. (A) Gel image after incubation of Pair A and Pair B (A, B), where either both sequences were modified with DBCO and azide (A1, B1) or only one was modified with DBCO (A2, B2). Only in sample A1 and B1 an additional band at higher size (A1: 1.4 kb, B1: 1.1 kb) can be observed, indicating a successful “click” reaction. (B) Gel image after fusion PCR in droplets. For Pair A, a slightly increased intensity is shown for the fused band (1.5 kb) in A1 compared to A2, while for Pair B, a significant increase in intensity of the fused fragment (1.2 kb) is detected in B1 compared to B2. Sample A3 and B3 correspond to pre-fused fragments, serving as positive controls. Sample C represents the negative control with water. (C) Gel image after nested PCR. Again, a higher band intensity is shown in sample A1 and B1 for both fused sequences (1.5 kb, 1.2 kb) compared to A2 and B2. PCR Ctrl+ represents already fused fragments. (D) Table of sample description. Samples A1-A3 (A1: both sequences modified, A2: one sequence modified, A3: already fused fragment) are referring to Pair A while samples B1-B3 (B1: both sequences modified, B2: one sequence modified, B3: already fused fragment) indicate samples of Pair B. Sample C represents the negative control. (E) qPCR results after nested PCR for Pair A and B, displaying the CT-values of each sample. For Pair A and B, the CT values of sample 1 (A1, B1) are lower than those from sample 2 (A2, B2) confirming the higher concentration and thus successful enrichment of fused fragment compared to the non-interacting sequences.

FIG. 6: shows data published from Kuwabara S, et al. (2021) “Microfluidics sorting enables the isolation of an intact cellular pair complex of CD8+ T cells and antigen-presenting cells in a cognate antigen recognition-dependent manner.” PLOS ONE 16(6): e0252666 [FIG. 1A]. The data shows that high frequency of cellular complex formation is dependent on the specific interaction between T and APC cells. The cells were gated using FSC and SSC, following which the cellular complex formation was analyzed using a two-dimensional dot plot. CFSE and CMTMR double-positive fractions were derived from the cellular complex between OT-I and ovaAPC. This is a representative plot of three independent experiments.

FIG. 7: shows the result of a pre-purification procedure using HA tagged T7 phage. (A) shows a schematic representation of the experimental setup. (B) shows the result of a quantitative PCR in cT values. Higher cT values indicate lower template concentration in the analysed samples samples (first bar indicates qPCR of T7 GFP phages of interaction experiment, second bar indicates qPCR of αGFP nanobody phages of interaction experiment, third bar indicates T7 GFP phages of control experiment, and fourth bar indicates qPCR of JUN of control experiment. (C) shows agarose gel of amplification products of nested PCR (3 samples are shown: DD-water control, T7 GFP (Bac)+T7 GFP nanobody (Mam) and T7 GFP (Bac)+T7Jun (Mam).

FIG. 8: shows a pre-purification of interaction partners before applying the method of the invention.

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

The examples show:

### Example 1: Screening Using Protein-Nucleic Acid Conjugates or Phage Display

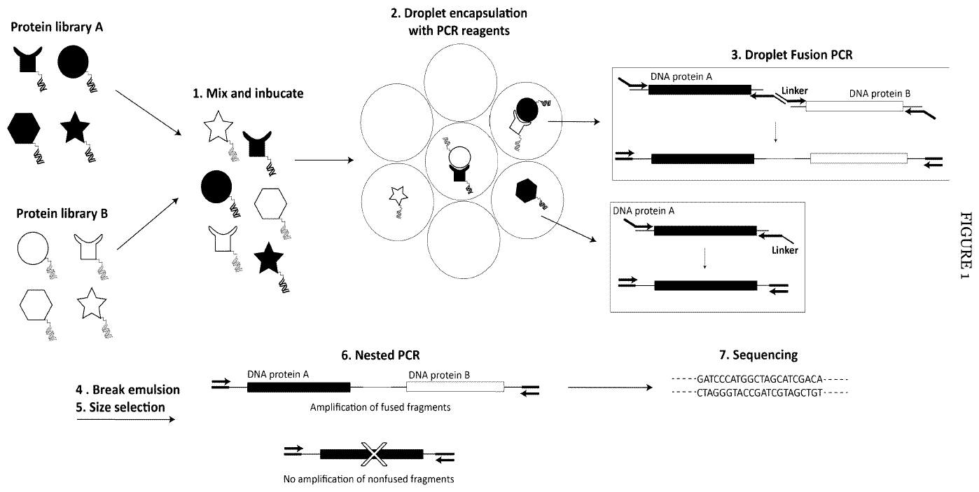

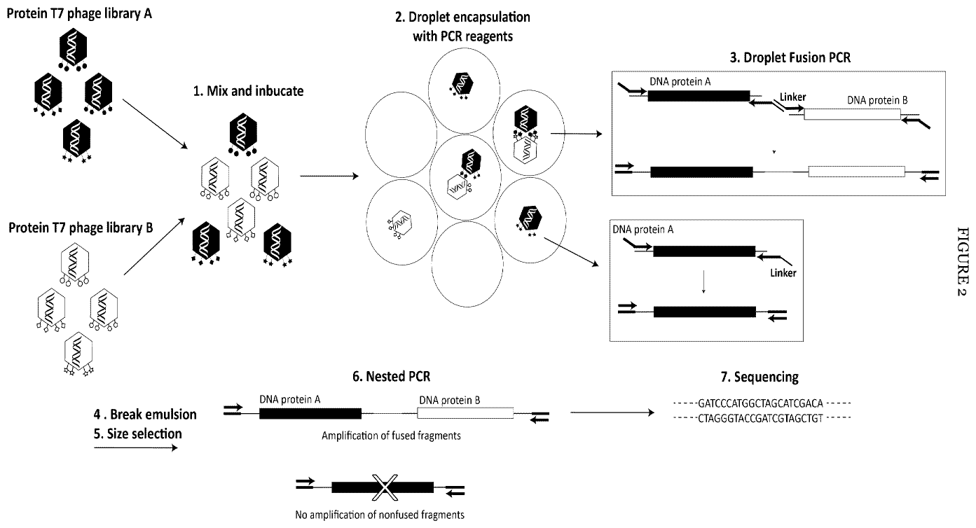

FIG. 1 shows an implementation of the invention screening for protein-protein interactions using a protein fused to a labelling (or barcoding) nucleic acid. In a first step two libraries are mixed with each other under conditions that allow for a formation of a binding interaction. The mixture is then encapsulated under conditions that provide restriction that not more than one entity or complexed entities is encapsulated within one droplet or compartment on average. In the final steps 3 to 7, a fusion PCR is performed within the compartment which subsequently allows for the identification of the presence and identity of interacting proteins.

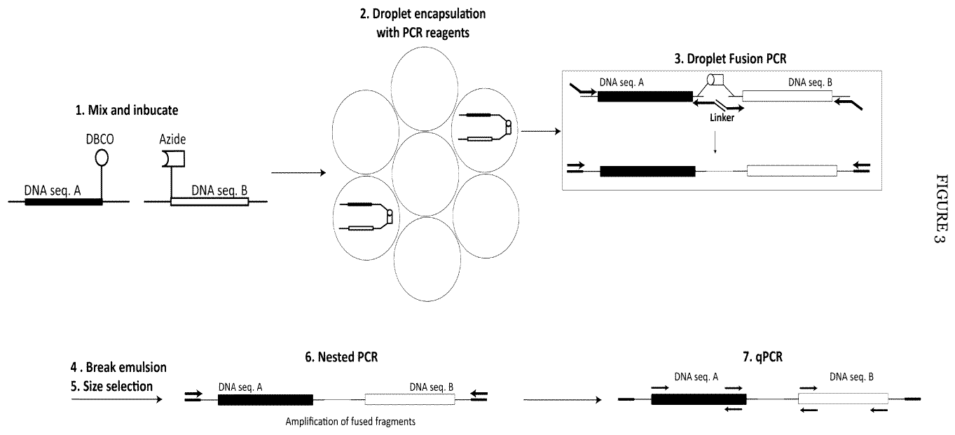

FIG. 2 shows an implementation using a phage display instead of protein conjugates.

As a model system for screening interacting versus non-interacting molecules, which also show an amplifiable genotype, oligonucleotide sequences modified with chemical reactive groups, based on a copper-free click chemistry reaction (FIG. 3) were utilized. These chemical groups form a covalent bond upon interaction, following a strain-promoted alkyne azide cycloaddition (SPAAC).

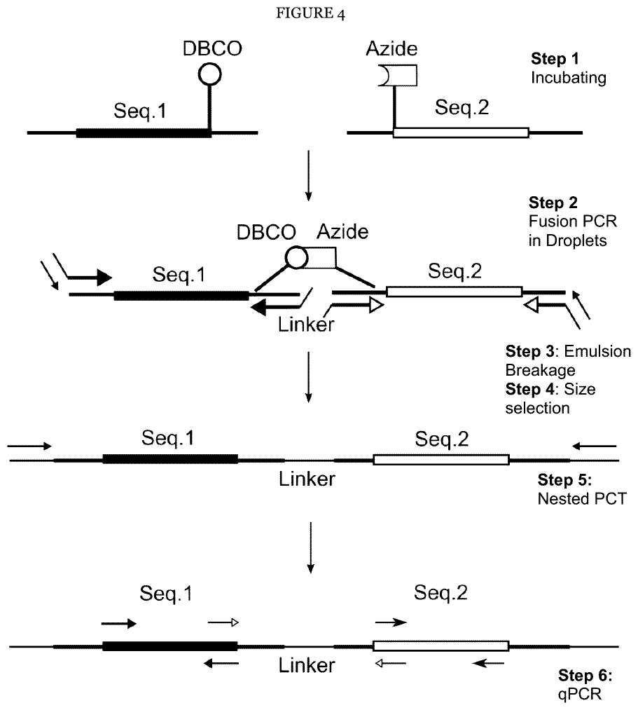

Here, sequences were modified with either a DBCO (=Dibenzylcyclooctyne), also known as ADIBO (=Azadibenzocyclooctyne) or DIBAC (=Dibenzoazacyclooctyne), -group or an Azide modification that “click” with DBCO, when incubated together, and thereby modelling an interaction. In this model system, the inventors generated sequences with and without such modifications, incubated and screened them for interaction versus no interaction in high-throughput utilizing droplet-based microfluidics. For the screen, the samples were diluted to a concentration that only one entity (either interacted pair or single, unreacted fragment) was finally found in the droplets. In the droplets, bound sequences were linked and amplified through fusion PCR. Upon emulsion breakage and size exclusion steps, the fused fragments were enriched by nested PCR and analyzed by gel electrophoresis and qPCR (FIG. 4).

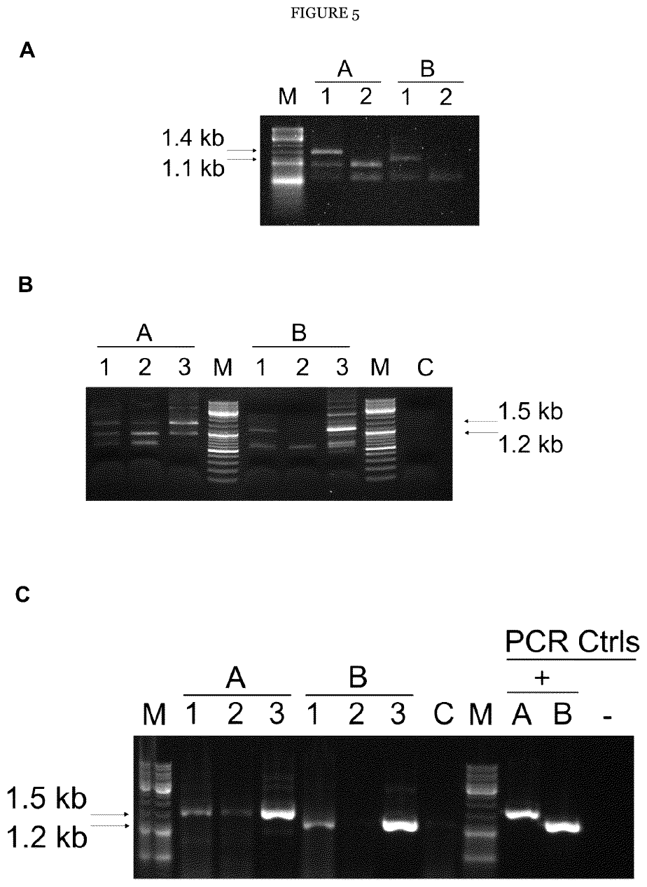

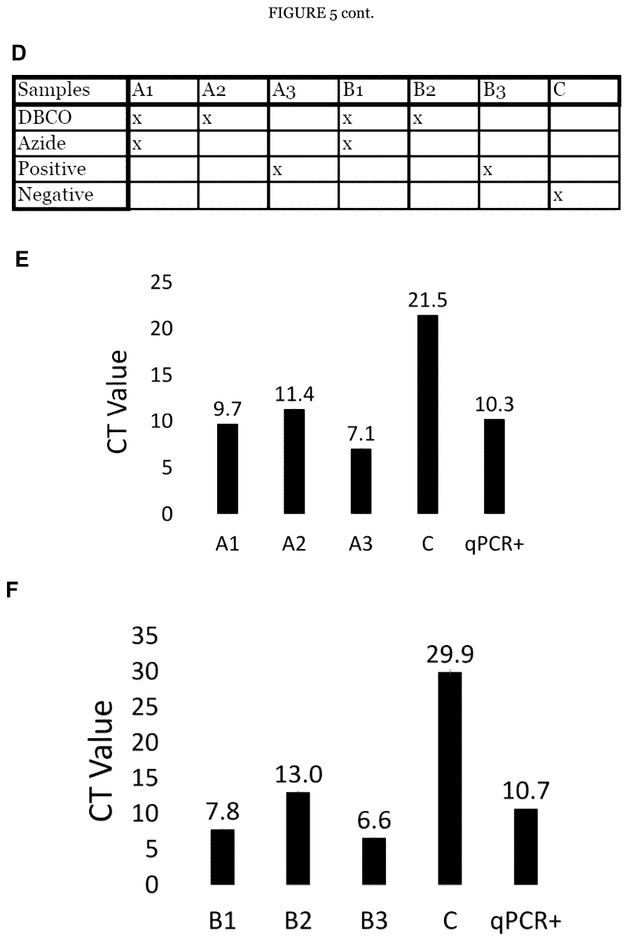

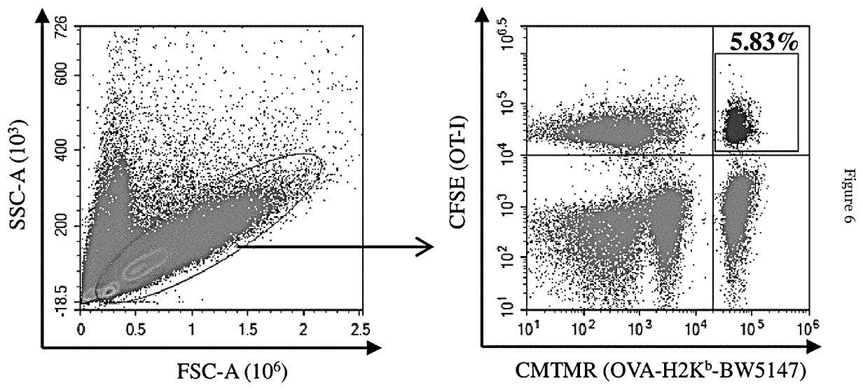

The inventors tested the model system with four different DNA sequences, resulting in two test pairs (Pair A+B). For both pairs, the first sample contained DBCO and Azide modified sequences (A1, B1). The second sample consisted of one modified (with DBCO) and one unmodified sequence (A2, B2). As a positive control, we also ran a third sample consisting of an already fused fragment (A3, B3). A water sample (C) served as a negative control (FIG. 5, D).

After incubation (FIG. 5, A) a band in higher size was only observed in sample A1 (˜1.4 kb) and B1 (˜1.1 kb), indicating that only with both modifications a “click” reaction was possible and that only there the binding of the two sequences was successful. After fusion-PCR in droplets (FIG. 5, B) and subsequent nested PCR (FIG. 5C), a higher band intensity was detected again in sample A1 (˜1.5 kb) and B1 (˜1.2 kb) compared to A2 and B2, respectively, showing that amplification and enrichment of the interacted, fused fragments was accomplished more successfully in those samples. The same trend can be observed in the qPCR results as well (FIG. 5, E). There the CT-values of the interacted samples (A1: 9.7, B1: 7.8) are lower than the corresponding counterpart (A2: 11.4, B2: 13). In conclusion, with this model screen, we successful observed a higher enrichment of the interacting sequences after fusion PCR and nested PCR while this was only shown to a lower extent for the non-interacting sequences, proving that this model-system is capable of distinguishing interaction vs non-interaction.

### Example 2: Screening Interactions of T-Cells and Antigen Presenting Cells

The method of the invention can be used to identify cell-cell interaction such as matching pairs of T-cell receptors (TCRs, displayed on T-cells) and antigens (displayed on the MHC2 complex of antigen presenting cells, APCs). While the potential use of yeast (=eukaryotic cell) display libraries had already been mentioned above.

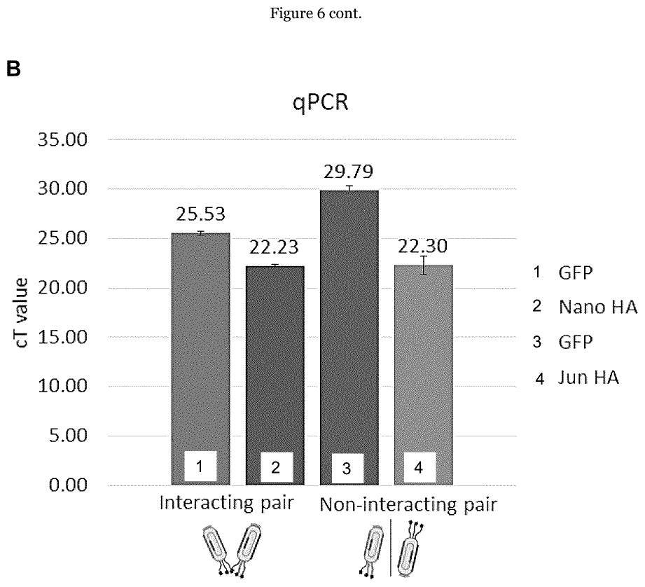

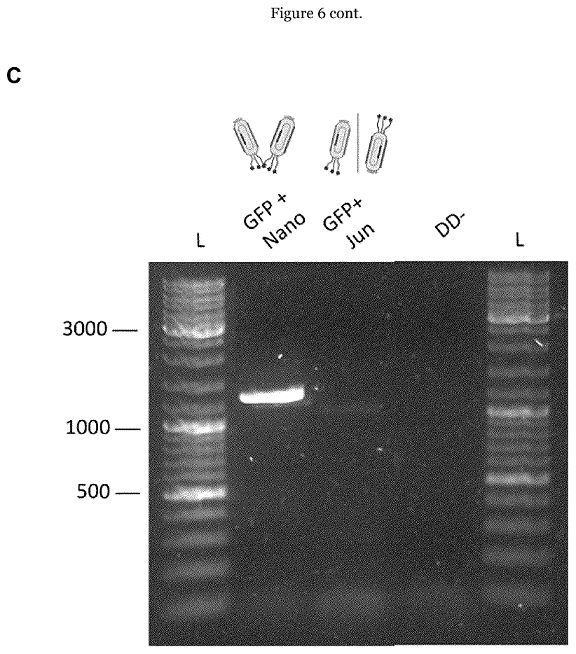

To study antigen-specific T/APC cellular interaction and complex formation at the cellular level, Kuwabara S et al. 2021 (see legend of FIG. 6 and references below) used splenocytes from OT-I mice in Rag1 knockout background and C57BL/6J WT mice as the antigen-specific and non-specific T cells, respectively. CD8+ T cells from OT-I transgenic mice recognized OVA-derived peptide SIINFEKL (OVA257-264) bound to H-2Kb of the MHC class I molecule. As an antigen non-expressing APC, Kuwabara S et al. 2021 used the previously established H-2Kb-expressing BW5147 cell line (H2Kb-BW5147). As OVA antigen-expressing APC, Kuwabara S et al. 2021 used both OVA- and H-2Kb-expressing BW5147 cells, which were generated as described in the method section (see Methods of Kuwabara S et al. 2021, incorporated herein by reference). To differentiate these cells, OVA-H2Kb-BW5147 (ovaAPC), H2Kb-BW5147 (nullAPC), OT-I, and C57BL/6 (WT) splenocytes were stained with the fluorescent dyes CMTMR, CMAC, CFSE, and Far Red, respectively. Stained cells of each cell type (1×10⁶) were mixed, followed by analysis of the percentage of CFSE/CMTMR (OT-I/ovaAPC) complexes (which indicated antigen-specific T/APC interaction) using conventional flow cytometry. One of the representative experimental results demonstrated that OT-I/ovaAPC complexes were detected in 5.83% of the total analyzed cells (FIG. 6 right which corresponds to FIG. 1A; right in Kuwabara S et al 2021). Hence, FIG. 6 shows data from a publication supporting the idea that cell-to-cell interactions between APCs and T-cells is sufficient to form duplets in a FACS assay, and that such duplets can be sorted. The data of the publication therefore indicates that such cell-to-cell interactions are applicable for a screening in accordance with the present invention. This is further supported by Giladi A et al. Nat Biotechnol 2020 May; 38(5):629-637) who show an analysis of T cell-dendritic cell (DC) pairs.

When screening for pairs of matching T-cells and APCs, in the event one only knows the sequence environment (to design primers for in droplet fusion-PCR) of one of the interaction partners (the TCR or BCR), the sequence of the antigen is typically not in a known fixed genetic location. Therefore, the screens may be conducted using either genetically modified antigen-presenting cells (in which the antigen is encoded in a known genetic content, e.g. using recombinant expression vectors with specific, known primer binding sites, or alternatively cells expressing peptide-MHC-I fusion proteins in which the presented antigenic peptide is encoded within the presenting cell directly). Alternatively, one can use an indirect labelling in accordance with the invention in which the second cell type is labelled with hash tag antibodies (antibodies binding common epitopes expressed on pretty much any cell and having a specific oligonucleotide barcode and therefore also known primer binding sites for the in-droplet fusion PCR). In the second case, one would still not be able to identify the displayed antigen, but it would it is possible to identify all TCRs or BCRs having a binding interaction with any of the antigens displayed on the second cell type

Note that the same principles could be directly applied to select memory B-cells expressing antibodies to surface proteins on a second cell type. The higher affinity of the assayed (assumed) interactions, the better applicable in the method of the invention.

### Example 3: Pre-Purification of Interaction Partners Using Tags

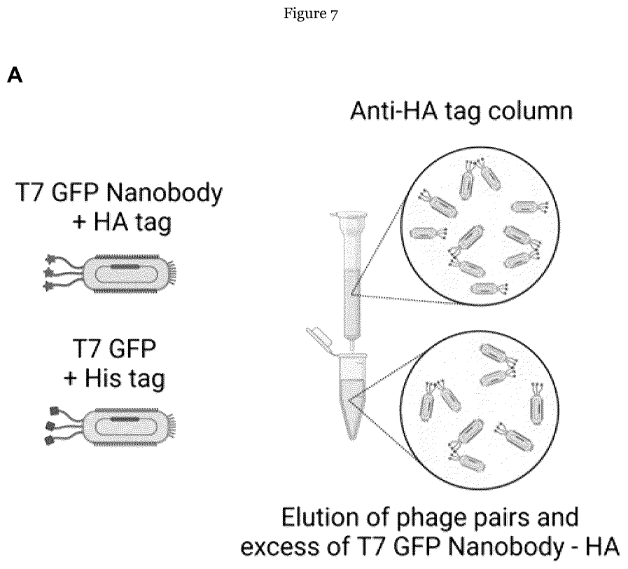

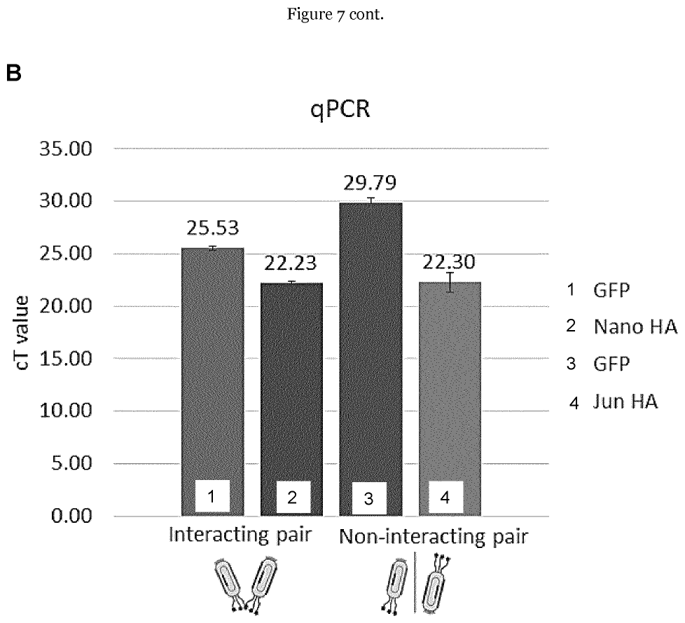

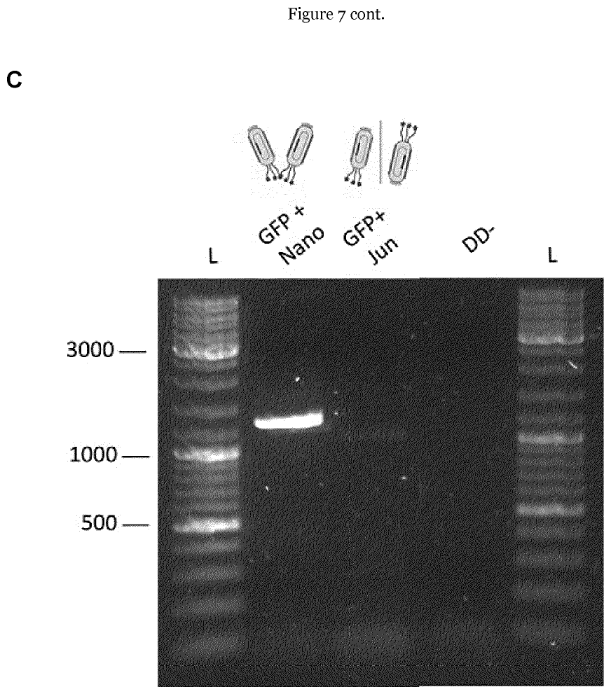

An experiment was carried out in which HA-tagged T7 phages displaying a GFP nanobody were incubated separately, either with non-tagged T7 phages displaying GFP (binding partner), or non-tagged T7 phages displaying the Jun protein (control). In both cases, the phage solutions were run over an HA column post incubation. This resulted in binding of the HA-tagged nanobody phages, while none of the two other phages should bind to the column on their own. However, due to the binding interaction between the HA-tagged nanobody phages and the GFP phages, one would expect a co-purification of the latter, resulting in the desired enrichment of binding partners. This was confirmed by qPCR measurements of the eluate fraction, after performing thorough washing steps. While the difference between the cT values of HA-tagged nanobody phages and the GFP phages was just 3.3 (indicating significant co-purification and hence interaction of the GFP phages), the difference between the cT values of the HA-tagged nanobody phages and the Jun phages was 7.49 (indicating no significant interaction). The basic experimental setup is shown in FIG. 7A, results provided in FIG. 7B.

Subsequently, the eluted phages were encapsulated into droplets at a density of less than 1 phage particle per 10 droplets and a fusion PCR was carried out in the emulsion format. Here, one would expect a strong band of the fusion product only for the interacting phages (nanobody and GFP) but not for the non-interacting phages (nanobody and Jun). Results are shown in FIG. 7C. As can be seen the band at the size of the fused fragment for interacting phages but only weak band for non-interacting phage pair. Hence, only DNA of interacting phage pairs get fused.

As a next step, an experiment was carried out in which four types of phages (HA-GFP, HA-39P15, Nano and RBD) were mixed directly at equal titers in a single tube. Only two of these phages (HA-GFP) and HA-39P15) were tagged with HA. Furthermore, only two (HA-GFP and Nano) should interact with each other. A fusion-qPCR specific for every possible fusion product (=fused genes of potentially interacting pairs) was carried out after different steps of the screening approach:
(a) Directly after bulk mixing of the four different phage clones.
(b) After pre-purification of binding partners using an anti-HA column
(c) After step 2) and additional encapsulation into droplets at a density of less than 1 phage particle per droplet.

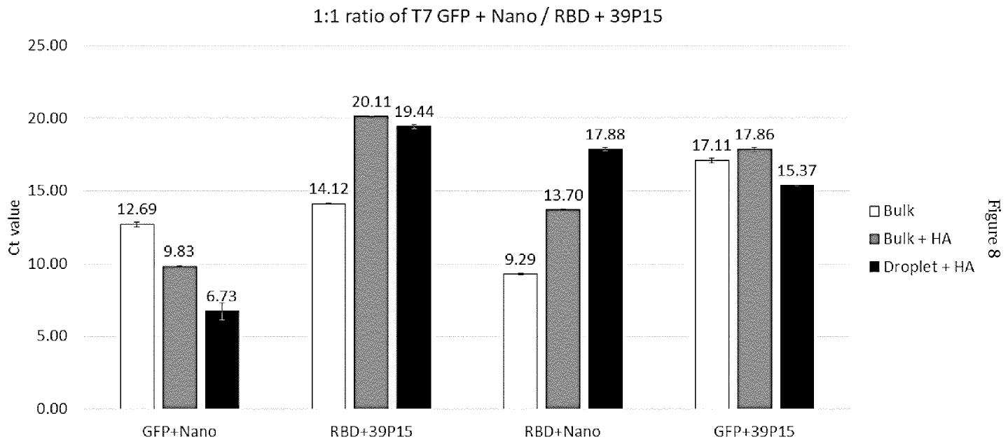

The results are shown in FIG. 8 and clearly show a specific enrichment of the interacting phage pair.

Following this workflow comprising steps 1-3, the cT values get smaller (=enrichment) only for the GFP-Nano fusion product, whereas for all other possible fusion product (of non-interacting pairs) they remain the same or even get larger. Noteworthy, the cT values of the interacting GFP-Nano pair further decreased (=showing further enrichment) during the droplet encapsulation step, showing that co-compartmentalization as described in the initial claim set still has a beneficial effect over simply using column purification. Furthermore, in case there would be more than just one single interacting pair in a screen, it would be impossible to separate them (amplify while maintaining the correct pairing) without droplet compartmentalization.

### REFERENCES

The references are:
Cui, N., Zhang, H., Schneider, N., Tao, Y., Asahara, H., Sun, Z., Cai, Y., Koehler, S. A., De Greef, T. F. A.,
Abbaspourrad, A., Weitz, D. A., & Chong, S. (2016). A mix-and-read drop-based in vitro two-hybrid method for screening high-affinity peptide binders. Scientific Reports, 6(March), 1-10. https://doi.org/10.1038/srep22575
Egloff, P., Zimmermann, I., Arnold, F. M., Hutter, C. A. J., Morger, D., Opitz, L., Poveda, L., Keserue, H. A., Panse, C., Roschitzki, B., & Seeger, M. A. (2019). Engineered peptide barcodes for in-depth analyses of binding protein libraries. Nature Methods, 16(5), 421-428. https://doi.org/10.1038/s41592-019-0389-8
Ledsgaard, L., Kilstrup, M., Karatt-Vellatt, A., McCafferty, J., & Laustsen, A. H. (2018). Basics of antibody phage display technology. Toxins, 10(6). https://doi.org/10.3390/toxins10060236
Mateus, A., Kurzawa, N., Becher, I., Sridharan, S., Helm, D., Stein, F., Typas, A., & Savitski, M. M. (2020). Thermal proteome profiling for interrogating protein interactions. Molecular Systems Biology, 16(3), 1-11. https://doi.org/10.15252/msb.20199232
Song M. & Hwang G. T. DNA-Encoded Library Screening as Core Platform Technology in Drug Discovery: Its Synthetic Method Development and Applications in DEL Synthesis J. Med. Chem. 2020, 63, 6578-6599.
https://doi.org/10.1021/acs.jmedchem.9b01782
Schubert, O. T., Röst, H. L., Collins, B. C., Rosenberger, G., & Aebersold, R. (2017). Quantitative proteomics: Challenges and opportunities in basic and applied research. Nature Protocols, 12(7), 1289-1294. https://doi.org/10.1038/nprot.2017.040
Younger, D., Berger, S., Baker, D., & Klavins, E. (2017). High-throughput characterization of protein-protein interactions by reprogramming yeast mating. Proceedings of the National Academy of Sciences of the United States of America, 114(46), 12166-12171.
https://doi.org/10.1073/pnas.1705867114
Yu, H., Tardivo, L., Tam, S., Weiner, E., Gebreab, F., Fan, C., Svrzikapa, N., Hirozane-Kishikawa, T., Rietman, E., Yang, X., Sahalie, J., Salehi-Ashtiani, K., Hao, T., Cusick, M. E., Hill, D. E., Roth, F. P., Braun, P., & Vidal, M. (2011). Next-generation sequencing to generate interactome datasets. Nature Methods, 8(6), 478-480. https://doi.org/10.1038/nmeth.1597
Giladi A, Cohen M, Medaglia C, Baran Y, Li B, Zada M, Bost P, Blecher-Gonen R, Salame T M, Mayer J U, David E, Ronchese F, Tanay A, Amit I. Dissecting cellular crosstalk by sequencing physically interacting cells. Nat Biotechnol. 2020 May; 38(5):629-637. doi: 10.1038/s41587-020-0442-2. Epub 2020 Mar. 9. PMID: 32152598.
Kuwabara S, Tanimoto Y, Okutani M, Jie M, Haseda Y, Kinugasa-Katayama Y, et al. (2021) Microfluidics sorting enables the isolation of an intact cellular pair complex of CD8+ T cells and antigen-presenting cells in a cognate antigen recognition-dependent manner. PLOS ONE 16(6): e0252666. https://doi.org/10.1371/journal.pone.0252666

1. A method for the identification of at least two interacting entities comprised in at least two separated libraries of candidate interacting entities, the method comprising the steps of:
(a) Providing at least a first candidate entity library and a second candidate entity library each library comprising a plurality of candidate interacting entities each of which is composed of at least an interacting-portion and a labelling-portion, wherein one or more candidate interacting entities of the first candidate entity library are assumed to interact with one or more candidate interacting entities of the second candidate entity library (and vice versa);
(b) Bringing into contact the candidate interacting entities of the first candidate entity library with the candidate interacting entities of the second candidate entity library under conditions that allow for the formation of an interaction-complex between at least two interacting entities;
(c) Encapsulating any entity and any interaction-complex from (b) in a plurality of microfluidic compartments under at least the conditions:
(i) A ratio of entities to compartments which is larger than 0 and less 1, and preferably is about 0.1; and
(ii) Optionally, a presence of one or more means for identifying of one or more labelling portion encapsulated within a compartment;
(d) Detecting subsequent to step (c) within the plurality of compartments, which comprise encapsulated entities, a presence of, and preferably an identity of, at least two labelling-portions encapsulated within a single compartment, wherein the presence of two labelling portions within a single compartment is indicative for an interaction between the two candidate interacting entities encapsulated within said compartment.

2. The method of claim 1, wherein the interacting-portion is selected from the group consisting of a polypeptide, peptide, glycoprotein, a peptidomimetic, an antibody or antibody-like molecule; a nucleic acid such as a DNA or RNA, a peptide nucleic acid (PNA), a carbohydrate such as a polysaccharide or oligosaccharide, including variants or derivatives thereof; a lipid such as a fatty acid and the like, including variants or derivatives thereof; or a small organic molecules including but not limited to small molecule ligands, small cell-permeable molecules, and peptidomimetic compounds.

3. The method of claim 1, wherein the labelling portion of each distinct candidate interacting entity comprises a nucleic acid molecule having at least one identification-sequence unique to the interacting-portion of the distinct candidate interacting entity (DNA-encoding library or DEL).

4. The method of a claim 3, wherein the identification sequence is flanked by an upstream primer binding sequence and a downstream primer binding sequence, which both are different and do not anneal to each other during an annealing phase of a PCR amplification cycle.

5. The method of claim 3, wherein the identification sequence comprises a nucleic acid sequence encoding at least parts of the amino acid sequence of the proteinaceous interacting-portion.

6. The method of claim 4, wherein each primer binding sequence of the labelling portion of the candidate interacting entity of the first candidate entity library differs from each primer binding sequence of the labelling portion of the candidate interacting entity of the second candidate entity library (and vice versa).

7. The method of claim 1, wherein step (d) involves a PCR amplification, preferably a fusion PCR and wherein the means for identifying of one or more labelling portion comprises components sufficient for conducting the PCR amplification, preferably the fusion PCR.

8. The method of claim 7, wherein the means comprise a first and a second PCR primer pair, wherein the upstream primer of the first PCR primer pair anneals to the upstream primer binding sequence of each labelling-portion contained in the first candidate entity library, and the downstream primer of the first PCR primer pair anneals to the downstream primer binding sequence of each labelling-portion contained in the first candidate entity library; and wherein the upstream primer of the second PCR primer pair anneals to the upstream primer binding sequence of each labelling-portion contained in the second candidate entity library, and the downstream primer of the second PCR primer pair anneals to the downstream primer binding sequence of each labelling-portion contained in the second candidate entity library.

9. The method of claim 8, wherein the upstream- and the downstream primer of the first primer pair comprises a first cross-hybridization sequence and the upstream- and the downstream primer of the second primer pair comprises a second cross-hybridization sequence; wherein the first- and the second hybridization sequence hybridize to each other under annealing conditions during a PCR annealing step.

10. The method of claim 9, wherein a PCR amplification in step (c) comprises a fusion PCR immediately followed by the removal of residual primer oligonucleotides and the subsequent nested PCR using (i) an upstream primer which anneals to the upstream primer binding sequence of each labelling-portion contained in the first candidate entity library, and a downstream primer which anneals to the downstream primer binding sequence of each labelling-portion contained in the second candidate entity library; or (ii) an upstream primer which anneals to the upstream primer binding sequence of each labelling-portion contained in the second candidate entity library, and a downstream primer which anneals to the downstream primer binding sequence of each labelling-portion contained in the first candidate entity library; wherein step (d) involves the detection of the amplification product of the nested PCR and wherein the presence of an amplification product indicates the presence of two labelling portions within a single compartment.

11. The method of claim 10, further comprising sequencing the amplification product of the nested PCR in order to determine the identity of the interacting-portions which were comprised within one compartment.

12. The method of claim 1, wherein a compartment is a droplet.

13. The method of claim 1, wherein in step (d) a detection of a presence of, and preferably an identity of, at least two labelling-portions encapsulated within a single compartment, involves a step of ligation of two labelling portions, and a subsequent step of detection of the presence of a ligation product.

14. The method of claim 13, wherein the labelling portions are nucleic acids, and the ligation involves a blunt or overhang ligation of the nucleic acid labelling portions.

15-36. (canceled)
